(19) United States
(12) Patent Application Publication
(10) Pub. No.: US 2004/0162519 A1
(43) Pub. Date: Aug. 19, 2004

(54) Aortic occlusion balloon cannula

(76) Inventors: Richard A. Helkowski, Redwood City, CA (US); Ivan Sepetka, Los Altos, CA (US)

Correspondence Address: John K. Uilkema, P.O. Box 190187, San Francisco, CA, 94119-0187, US

(21) Appl. No.: 10/778,778

(22) Filed: Feb. 12, 2004

Related U.S. Application Data
(63) Continuation of application No. 09/299,888, filed on Apr. 27, 1999, now abandoned.

(51) Int. Cl. A61M 31/00

(52) U.S. Cl. 604/103.09

(57) ABSTRACT

A multi-lumen cannula device is provided having an occlusion balloon for partitioning a vessel, such as the aorta during a CPB procedure. The cannula has a first lumen for delivering fluid to a location distal to the balloon, a second lumen for delivering fluid to a location proximal to the balloon, and an inflation lumen through which the balloon may be inflated. In certain embodiments, the cannula has a flexible proximal portion and a more rigid distal portion maintained at an angle thereto. The distal and proximal structures of the balloon facilitate reliable balloon positioning and occlusion within the vessel.

### FIELD OF THE INVENTION

The present invention relates generally to surgical instruments, and more particularly to a multi-lumen cannula having an occlusion balloon for partitioning a vessel, such as the aorta during a cardiopulmonary bypass procedure.

### BACKGROUND OF THE INVENTION

In certain cardiac surgical procedures, it is desirable to arrest the heart for an extended period of time. An arrested heart provides the surgeon with a motionless, decompressed heart and a relatively dry, bloodless operating field. While the heart is arrested, however, the life supporting functions of the heart and lungs must be provided by alternate means. Typically, the heart and lungs of the body are bypassed by way of a conventional cardiopulmonary bypass (CPB) system.

In a basic CPB system, oxygen-poor blood is drained by means of gravity or siphoned from the patient's venous circulation and is transported to a pump-oxygenator, commonly known as the heart-lung machine, where the blood is exposed to a gaseous mixture that eliminates carbon dioxide and adds oxygen to the blood. The oxygenated blood is then returned or perfused into the patient's arterial circulation for distribution throughout the entire body. This process requires a venous drainage cannula (or cannulae) to be placed into the right side of the heart (typically the right atrium) or directly in the major veins (typically the superior vena cava and/or the inferior vena cava or through peripheral vein access sites to drain unoxygenated blood from the patient and deliver it to the heart-lung machine. Similarly, an arterial or aortic perfusion catheter or cannula is placed in the aorta or another large peripheral artery, such as the common femoral artery, to return or perfuse oxygenated blood to the patient. The heart and lungs of the person is thereby effectively bypassed.

Once CPB has been established, cardioplegia is used to arrest the beating of the heart, and in some procedures, to provide oxygen or protective solutions to the myocardium. Cardioplegia is administered by delivering a cardioplegic solution, such as potassium, magnesium, procaine, or a hypoclacemic solution, to the myocardium. Cardioplegia may be administered antegrade by infusing cardioplegic fluid through the coronary arteries in the normal direction of blood flow or retrograde by infusing cardioplegic fluid directly into the coronary sinus where it then flows backwards into the capillaries and coronary arteries of the myocardium.

In one common CPB configuration used to provide arterial perfusion and/or antegrade delivery of cardioplegic solution the ascending aorta is cross-clamped in the area between the brachiocephalic artery and the coronary ostia. The cross-clamp partitions the aorta into an downstream portion leading to the brachiocephalic, left carotid, and left sub-clavian arteries and an upstream portion which includes the region of the aortic root and the coronary ostia. A coronary perfusion cannula may be inserted through an incision downstream of the cross-clamp to supply oxygenated blood to the body. A cardioplegia cannula having a distally extending needle or obturator may be inserted into the aorta upstream of the cross-clamp to inject cardioplegic solution into the aortic root where it drains in the normal direction of blood flow into the coronary ostia, through the coronary arteries, and into the capillaries within the myocardium. Often the cardioplegia cannula is used to vent the ascending aorta to prevent air embolism as the cross-clamp is removed after completion of the surgery.

Conventional external cross-clamps typically include opposing jaws connected to an elongated shaft having handles at the proximal end for controlling the positioning and actuation of the clamp jaws. Such external cross-clamping devices tend to hinder the surgeon's access to the operating site. More importantly, crushing the aorta closed with external jaws tends to cause damage to the aorta itself as well as greatly increases the likelihood that plaque or calcifications will become dislodged from the aortic walls. Material dislodged from the periphery of the aortic wall may proceed directly to the brain causing severe cerebrovascular complications (i.e., stroke). For patients having a highly diseased, hardened, or calcified aorta, the risk associated with external cross-clamping may be too great to warrant its use except in the most exigent circumstances.

Instead of an external cross-clamp, it is well known that a large aortic occlusion balloon could be used to occlude or partition the aorta to facilitate the CPB procedure. For example, EP 0 218 275 A1, published. Apr. 15, 1987 and having the title “MULTI-PURPOSE CATHETER”, describes a multi-lumen catheter device having a balloon for partitioning the ascending aorta between the coronary ostia and the brachiocephalic artery, a lumen for delivering blood from the heart-lung machine to a downstream side of the balloon, and lumen for transporting cardioplegic or other fluids to an upstream side of the balloon so that it reaches the coronary arteries.

Although the use of an intra-aortic balloon to occlude the aorta in place of an external clamp is believed to significantly reduce the incidence of damage to the aortic tissue and reduce the occurrence of dislodged plaque or calcified material, there remains a number of problems. For example, since the intra-aortic occlusion balloon must be inflated while the heart is still beating, it is difficult to maintain the balloon in the desired position as it inflates against the pulsating blood flow from the heart. This is especially true for catheters which may require distal tips having sufficient flexibility to be delivered through a peripheral artery or vein, such as the femoral artery. As the balloon inflates, any shifting or positional displacement of the balloon may result in the balloon becoming anchored in an undesirable position or orientation.

Another problem is that the occlusion balloons often exert excessive localized pressure against the interior wall or the aorta. Such high pressures arise primarily from the balloon's requirements to achieve complete occlusion and resist positional migration. Complete balloon occlusion of the aorta is often difficult without resorting to potentially damaging, high balloon inflation pressures, especially when balloon inflation is compounded by any significant misorientation. In addition, once the balloon is fully inflated to occlude the aorta, the differential pressures encountered by the balloon during CPB and cardioplegia delivery tend to displace the balloon, resulting in leakage of fluid past the occlusive balloon or migration of the balloon to an undesirable location (i.e., blocking the brachiocephalic or coronary arteries, or interfering with the aortic valve).

In view of the foregoing, it would be desirable to have an intra-aortic occlusion balloon cannula for delivering oxygenated blood and cardioplegic fluid to the aorta which is reliably positionable for inflation within the aorta. It would be further desirable to have an intra-aortic occlusion balloon cannula having a balloon configuration that achieves complete and reliable occlusion and resists migration without transmitting excessive pressure to the aorta.

### SUMMARY OF THE INVENTION

The present invention will be described in the context of direct cannulation and occlusion of the aorta between the brachiocephalic artery and the coronary ostia for delivery of oxygenated blood and cardioplegic fluid during CPB, but the invention is not limited thereto. It is contemplated that the present invention may be a cannula adapted for use at other locations for delivering or for perfusing other fluids.

The present invention involves a cannula having an inflatable member for occluding or partitioning a vessel and one or more lumen for delivering fluid to the vessel. It is an object of the present invention to provide a cannula that allows reliable and stable positioning of the inflatable member during inflation and thereafter during use. Another object of the present invention is to provide a cannula which has an inflatable member adapted to achieve complete vessel occlusion at lower pressures.

One aspect of the present invention involves a cannula device for delivering oxygenated blood and cardioplegia to the aorta, preferably the ascending aorta between the brachiocephalic artery and the coronary ostia. The cannula device generally has an elongated tubular shaft or body having a proximal portion and a distal portion. A length or section of the distal portion may be curved or disposed at an angle relative to the longitudinal axis of the proximal portion.

Preferably, the angle between the distal section and the proximal portion is in the range of about 100 degrees to about 140 degrees, more preferably about 105 degrees to about 115 degrees, most preferably about 110 degrees. This particular angular relation between the distal section and the proximal section allows the distal section of the cannula to be easily and atraumatically inserted through an incision in the ascending aorta and tends to position the proximal end in such a manner as not to obstruct the surgical site.

A distensible or inflatable member is attached to the distal section. In use, inflatable member occludes the vessel at a desired location, partitioning the vessel into a downstream portion and an upstream portion. The cannula may have one or more lumen for delivering or removing fluid from each side of the balloon. In a preferred embodiment, the cannula device has a first lumen extending from the proximal portion to an open port distal to the balloon, a second lumen extending from the proximal portion to an open port proximal to the inflatable member, and an inflation lumen in fluid communication with the inflatable member.

In one embodiment of the present invention, the distal portion is substantially stiffer than the proximal portion. Preferably, the distal portion includes a substantially rigid support tube. The support tube may be made of a metal, such as stainless steel. The stiff distal section serves a number of purposes. The stiff distal section tends to support the inflatable member during positioning, support the inflatable member against the fluid pressures of the beating heart and against the perfusion pressures associated with CPB, support the first lumen against collapse under the pressure of the inflating member, and provides a desirable substrate for attachment of the inflatable member.

The proximal portion is preferably relatively flexible, and may include a coiled or braided wire or ribbon member along at least a portion of its length for improved kink-resistance. A flexible proximal portion may allow easy positioning and manipulation of the proximal portion within the surgical site and substantially prevents such movements from being transmitted to the distal section positioned within the vessel.

Another aspect of the present invention involves a cannula device for partitioning a vessel within a body into an upstream portion and a downstream portion. The cannula device has an elongated tubular body having a proximal portion and distal portion, the distal portion having at least a section thereof disposed at an angle relative to the proximal portion. An inflatable member, preferably made of a material which is capable of an elastic elongation of at least 500%, more preferably at least 700%, is attached to the proximal section. Such highly compliant balloons inflate with less pressure and tend to achieve occlusion of the vessel with minimal disturbance to the interior wall of the vessel.

In a preferred embodiment, the cannula has a first lumen having an open port distal to the inflatable member, a second lumen having an open port proximal to the inflatable member, and an inflation lumen in fluid communication with the inflatable member. The first lumen may be sized and adapted to deliver a sufficient amount of oxygenated blood to the ascending aorta which in turn feeds the brachiocephalic, left carotid, left subclavian and descending aorta. The second lumen may be sized and adapted to deliver cardioplegic fluid to the coronary ostia.

In a preferred embodiment, the inflatable member is an inflatable balloon which may be a molded or extruded tube. The inflatable balloon is preferably made of a highly compliant material, preferably silicone which exhibits an elastic elongation of at least 500%, more preferably at least 700%. The balloon may be attached to the cannula by any suitable bonding, welding, or adhesive technique.

In a preferred embodiment, the distal portion of the cannula has a substantially rigid support member having an inner surface, an outer surface, and a layer of biocompatible material disposed over at least a portion of the outer surface. In one embodiment, the balloon may be a distensible or expandable section of the layer itself. In another embodiment, the inflatable balloon is preferably attached to the exterior surface of the polymeric layer. In one embodiment, the balloon member is attached using an adhesive or the like. In another embodiment, the balloon is generally tube shaped and is attached at either or both of the balloon ends to the exterior surface using one or more ties, bands, or sutures. The sutures may be a single loop or may be several wraps or turns of suture material and are affixed in such a manner as to radially compress the balloon material into the polymeric layer and against the support member.

These and other advantages of the present invention will become apparent from the following detailed description in conjunction with the accompanying drawings and appended claims.

### BRIEF DESCRIPTION OF THE DRAWINGS

FIG. 1 is an intra-aortic occlusion balloon cannula constructed in accordance with the principles of the present invention.

FIG. 2 is a view along section line 2-2 as indicated in FIG. 1.

FIG. 3 is the distal end of the cannula of FIG. 1.

FIG. 4A is a preferred construction of an intra-aortic balloon cannula according to the principles of the present invention.

FIG. 4B is a partial cross-sectional of a ribbed balloon.

FIG. 5 is a balloon inflated within the aorta according to the present invention.

FIG. 6 is a highly compliant balloon inflated within the aorta.

FIG. 7 is the aortic occlusion cannula of the present invention.

FIG. 8 is an optional suture flange for securing the cannula to the aorta.

FIG. 9 is a suture flange fastened to an artery.

FIG. 10 is the intra-aortic balloon cannula of the present invention having a steerable tip to position the device within the aorta.

FIG. 11 is the intra-aortic balloon cannula having a steerable tip positioned within the aorta.

### DETAILED DESCRIPTION

The present invention involves a cannula for perfusing fluids within the vasculature of the body. The cannula has an inflatable balloon for occluding the flow lumen of a vessel and one or more ports for perfusing fluid into the vessel or for removing fluid from the vessel. In one embodiment, the present invention involves a cannula having one or more ports distal of the balloon for perfusing or removing fluid through a first cannula lumen and one or more ports proximal of the balloon for perfusing or removing fluid through a separate, second cannula lumen. Although the cannula of the present invention will have utility in a number of surgical procedures, for purposes of illustration only it will be described below primarily with reference to a CPB procedure.

One aspect of the present invention involves a cannula for direct insertion into a vessel through an incision made therein. The cannula generally has an elongated tubular body and a distal tip section, at least a portion of which is preferably disposed in an angular relation to the body. The cannula has an inflatable balloon located on the distal tip section for occluding the vessel lumen and preferably has at least a first lumen having at least one port proximal to the balloon, a second lumen having at least one port distal to the balloon and an inflation lumen for delivering an inflation fluid to the balloon. Although the present invention will be described below with reference to a cannula configured for insertion directly into a vessel, many of the features of the present invention will apply equally to catheters which access the target site percutaneously through the femoral or other peripheral vein or artery.

The structural properties of the various sections of the cannula is an important aspect of the present invention. For example, the distal section is configured to ensure reliable insertion and positioning of the inflatable balloon and perfusion ports. The proximal section of the cannula has flexible, kink-resistant configuration that allows the proximal section to be maneuvered and positioned in a manner to provide minimal obstruction to the surgical field and without affecting the position or orientation of the distal section.

In a preferred embodiment, the present invention involves a multi-lumen cannula having a distal occlusion balloon which may be inflated to partition a vessel, such as the aorta, into a downstream portion and an upstream portion. The cannula has a first lumen for delivering oxygenated blood from a CPB pump to the downstream portion and a second lumen for delivering fluids to the upstream portion. Preferably, the second lumen is configured to administer cold cardioplegia, tissue protective solutions, or other drugs or therapeutic agents to the upstream portion. The second lumen may also be used to vent the upstream portion.

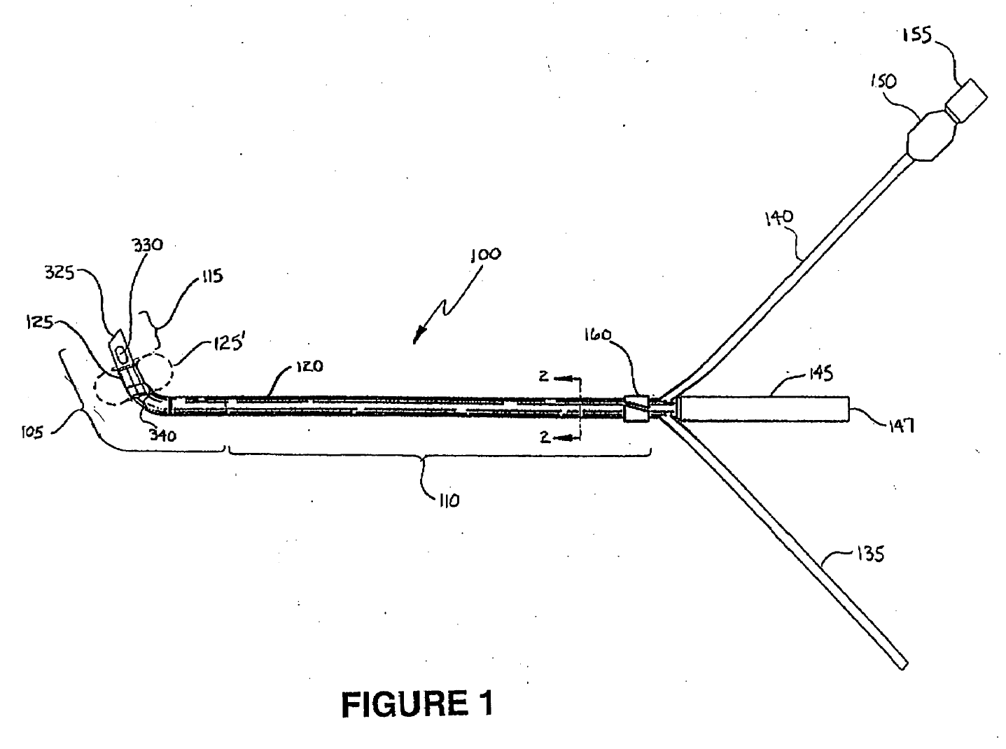

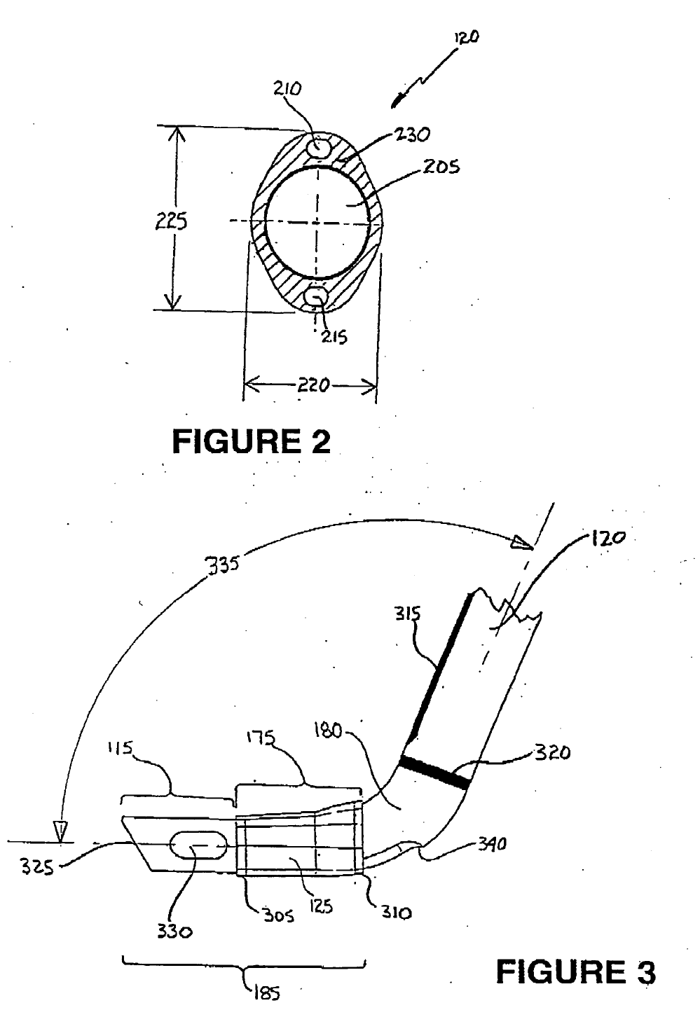

Referring to the drawings, wherein like numerals indicate like elements, FIGS. 1-3 illustrate the general features of a preferred cannula constructed according to the principles of the present invention. Cannula 100 has a multi-lumen elongate tubular body having an first lumen 205, a second lumen 215, and a balloon inflation lumen 210. Inflatable balloon 125, having an inflated condition 125′, is located on a distal section of cannula 100. Tip section 115 extends distally from the location of the balloon and preferably includes one or more perfusion ports. The preferred arrangement of first lumen 205, second lumen 215, and inflation lumen 210 results in a cannula body having a width 220 which is greater than the height 225, allowing for easy insertion through a small incision.

First lumen 205 has at least one opening or port distal to the balloon and sized and configured to allow perfusion of oxygenated blood. In one embodiment, first lumen 205 has an end port 325 and one or more side ports 330. First lumen 205 is connected proximally to inlet tube 145. The proximal end 147 of inlet tube 145 may be connected to a fluid source. Fluid supplied to inlet tube 145 flows through first lumen 205 and is perfused into the vessel from end port 325 and side ports 330.

Second lumen 215 has at least one opening or port 340 proximal to the balloon. At its proximal end, second lumen 215 is connected to second inlet tube 135, the proximal end of which may be connected to a second fluid source. Fluid supplied to second inlet tube 135 flows through second lumen 215, exiting from port 340.

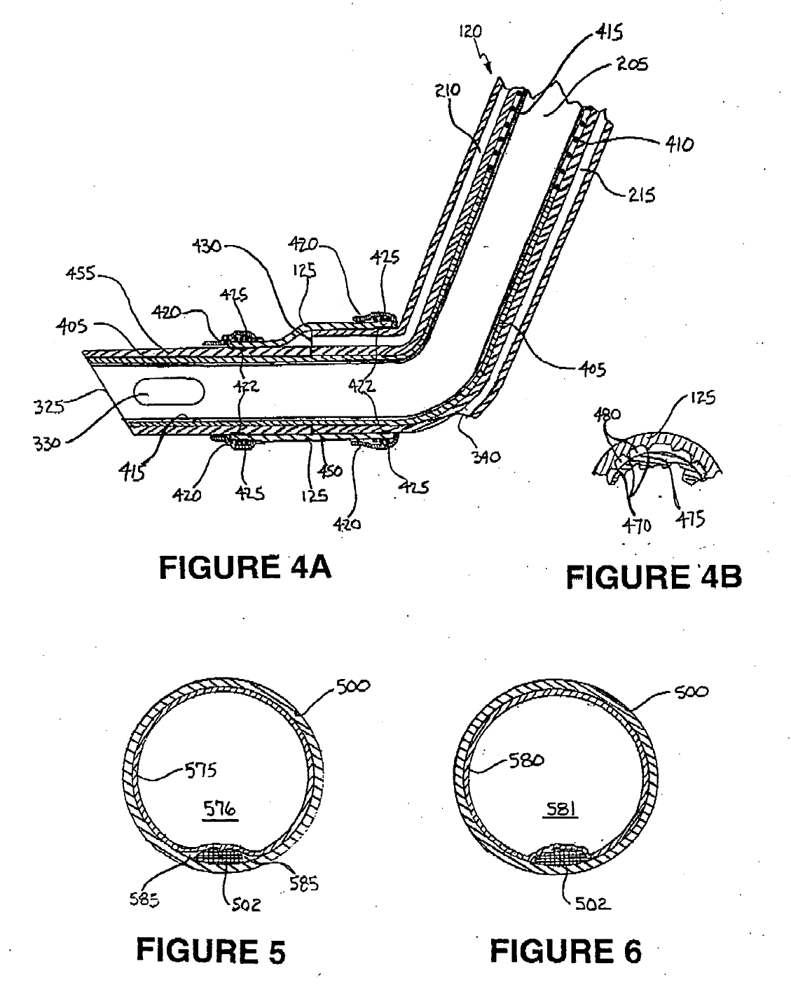

Inflation lumen 210 has at least one opening or port 430 (see FIG. 4) in fluid communication with the interior of inflatable balloon 125. The proximal end of inflation lumen 210 is connected to inflation inlet tube 140, the proximal end of which is preferably connected to a source of inflation fluid.

In a preferred embodiment, inlet tube 140 includes a pilot balloon 150 and connector 155 which may be valved. Pilot balloon 150 is an expandable or inflatable balloon member which serves a number of functions. First, pilot balloon 150 tends to regulate the pressure of the inflation fluid as it is injected through connector 155. In addition, once balloon 125 has been inflated to the desired expanded condition 125′, pilot balloon 150 tends to regulate the pressure in balloon 125 in response to external loads applied to the balloon. Thus, in use, an external force encountered by balloon 125 in its inflated condition 125′ would simply cause pilot balloon 150 to expand somewhat, instead of resulting in an increased pressure solely within the interior space of the balloon 125 and an increased stress in the material of balloon 125. Finally, as the inflation fluid is withdrawn (typically under vacuum) to deflate balloon 125, pilot balloon 150 provides a visual indication when balloon 125 has been completely deflated by itself collapsing to a substantially completely deflated state. This visual indication is important because an attempt to withdraw a balloon that is not fully deflated from the aorta could severely tear or otherwise damage the incision in the aorta.

To facilitate optimal access to the internal lumen of a body vessel, cannula 100 preferably has a distal end configured at an angle to the longitudinal axis of main body section 120. In the preferred embodiment shown, cannula 100 has distal body section 185 curved or angled in relation to main body section 120. In a preferred embodiment, distal body section 185 is substantially straight to correspond to the axis of the body vessel lumen to which it will be inserted. Distal body section 185 preferably has sufficient length to accommodate balloon 125 and tip section 115. Transition section 180, typically curved or radiused, connects main body section 120 with distal body section 185.

When cannula 100 is configured for placement in the ascending aorta between the brachiocephalic artery and the coronary ostia, angle 335 between main body section 120 and distal body section 185 is preferably about 100 degrees to about 140 degrees, more preferably about 105 degrees to about 115 degrees, most preferably about 110 degrees. This angular relationship tends to allow easy insertion of distal body section 185 through an incision in the ascending aorta and orients main body section 120 in a manner which tends not to obstruct the surgical site. This angular relationship also allows blood pumped from the heart to flow past that part of the cannula body upstream of the balloon and act on the balloon more symmetrically around the diameter of the balloon.

According to one embodiment of the present invention, the cannula is constructed to have regions which have different structural characteristics to support the various specialized functions of the cannula. In a preferred embodiment, cannula 100 has substantially rigid distal portion 105 that is less flexible than proximal portion 110. The more flexible proximal portion 110 may be conveniently manipulated and positioned without transmitting the associated forces to the distal portion inserted within the aorta.

Rigid distal portion 105, and the differential rigidity with less rigid proximal portion 110 provides a number of advantages. Among other things, rigid distal portion 105 provides for easy, reliable and atraumatic insertion of the instrument through an incision in the aorta and provides excellent tactile feedback from the instrument tip to the surgeon. Rigid distal portion 105 also provides greater support for balloon 125. The rigid distal portion 105 provides sufficient axial and flexural stiffness to allow the balloon to be held in the desired position and, in combination with the more flexible proximal portion 110 allows advantageous orientation for stability against the systolic and parastolic pressures of the beating heart, as well as the perfusion pressures encountered during CPB and cardioplegia delivery.

The differential structural properties of cannula 100 can be accomplished in a number of ways. For example, the desired structural characteristics of rigid distal portion 105 may be obtained by using a material of greater cross-section or higher flexural modulus or durometer than that of the proximal portion 110. In a preferred embodiment, rigid distal portion 105 includes a substantially rigid support member to supply the desired rigidity. This allows the various cannula portions to be made of the same or similar flexible, thin-walled material, eliminating the bonding, manufacturing, and reliability problems associated with materials that are dissimilar in formulation or cross-section. Because the desired distal rigidity is obtained largely from the support member and not the cannula material, thinner walls may be used leaving an in increased cross-sectional area available for fluid flow through the various lumen.

FIG. 4 illustrates a preferred construction for achieving the desired differential structural properties. The multi-lumen tubular body preferably has a substantially unitary seamless construction providing first lumen 205, second lumen 215, and a balloon inflation lumen 210. The cannula body itself may be made from a thin-walled, flexible, surgical grade polymer. Preferably, the cannula body material is selected from the group consisting of polyethylene, polyvinyl chloride, polyester, polypropylene, polyamide, polyurethane, polystyrene, fluorine plastics, silicone rubber, elastomers, and composites of the above. Most preferably the cannula body is made of an extrudable medical grade silicone.

Inside the cannula body material, distal portion 105 optimally includes a support member to increase the rigidity. The support member may be in the form of, for example, a substantially rigid braided wire or ribbon tubular material, one or more longitudinally extending plastic or metal stiffening members, or a polymeric or metallic tube or the like. In a preferred embodiment, rigid distal section 105 has support member 405 is in the form of a thin walled tube extending from tip section 105 to a location proximal to balloon 125. More preferably, support member 405 extends proximally a sufficient distance to enable the proximal end of rigid distal portion 105 to remain outside the vessel during use.

Support member 405 may be of any suitable biocompatible polymer or metal. Preferably support member 405 is made from stainless steel tubing having a thickness in the range of about 0.01 inches (0.25 mm) to about 0.05 inches (1.27 mm). Most preferably support member 405 is made from AISI 304 Stainless tubing having a wall thickness of about 0.01 inches (0.25 mm) to about 0.015 inches (0.38 mm). This particular tubular construction provides exceptional stiffness to the distal portion 105 and eliminates any possibility of first lumen 205 collapsing as a result of kinking or collapsing from the inward forces resulting from the pressure required to inflate balloon 125.

Inside cannula body material 230, proximal portion 110 may include a helically wound or braided wire or ribbon material to improve the kink resistance of proximal portion 110. In one embodiment, proximal portion 110 includes metallic coil 410. Coil 410 may be made from any suitable coil material including titanium, tantalum, stainless steel, and materials having super-elastic properties. In a preferred embodiment, coil member 410 is made from stainless steel wire (for example, AISI 302 stainless steel wire) having a diameter in the range from about 0.005 inches (0.127 mm) to about 0.030 inches (0.76 mm), most preferably about 0.01 inches (0.25 mm). Coil 410 provides a measure of kink resistance to proximal section 110, and more specifically to first lumen 205, while allowing proximal section 110 to remain relatively flexible.

As mentioned above, first lumen 205 may be sized and configured to deliver oxygenated blood from the CPB machine. For that purpose, it is important for interior lumen 205 to be smooth and continuous so as not to damage the oxygenated blood. Coating layer 415 may be used to cover support member 405 and coil 410, thus providing the desired surface characteristics for first lumen 205. In addition, coating layer 415 may extend somewhat in between the individual coils of coil 410, thus stabilizing the position of the coils as proximal portion 110 is flexed for positioning or otherwise manipulated. Coating layer 415 is preferably a thin layer of silicone. For purposes of illustration only, the cross-sectional area of first lumen 205 for delivering oxygenated blood to an adult human may be in a range from about 0.02 square inches (12.9 mm²) to about 0.20 square inches (129.03 mm²), more preferably in the range from about 0.04 square inches (25.81 mm²) to about 0.05 square inches (32.26 mm²).

Balloon 125 is preferably a thin expandable or inflatable member mounted directly onto the exterior of distal body section 185. The balloon may be mounted to the exterior of the cannula by any number of known techniques. In a preferred embodiment, balloon 125 is in the form of an extruded tube and is placed over distal body section 185 and bonded or otherwise attached at a distal location 305 and a proximal location 310 (see FIG. 3) thus creating a sealed space which may be inflated by a fluid delivered through inflation lumen 210 and inflation port 430.

At least in part because the rigid distal section 105 provides the necessary structure to reliably hold the balloon area in position during inflation and thereafter, balloon 125 may be made of a highly compliant material. Thus, when balloon 125 is mounted to and supported by rigid distal section 105, its primary function is to seal against the vessel wall, and balloon 125 is not required to bear the structural requirements relating to resisting positional migration or maintaining the orientation of the distal perfusion ports.

In one embodiment, the balloon material may be made from a material which exhibits at least 500% elongation, that is a section of material may be stretched to more than 5 times its original length and, upon release of the stress, will return with force to its approximate original length. Preferably, balloon 125 is made of a material which can undergo elongation in the range of about 700% to about 900%, more preferably about 800% or more. Most preferably, balloon 125 is made from a medical grade silicone capable of elongation of about 800% (such as NUSIL 4025, commonly available from Nusil Technology of Carpinteria, Calif.). The balloon may have a thickness in the range of about 0.015 inches (0.381 mm) to about 0.030 inches (0.762 mm), more preferably about 0.02 inches (0.508 mm).

To keep the central (non-bonded) region of the highly compliant balloon material from becoming adhered or otherwise stuck to the exterior surface of the cannula to which it is mounted, the interior surface of balloon 125 may be textured or roughened or have interior raised features that hold at least a portion of the balloon material away from the exterior mounting surface. FIG. 5 illustrates balloon 125 having a number of longitudinal internal ribs 470 creating a number of spaces 480 between an exterior surface 475 and balloon 125. The raised features or ribs are preferably on the order of about 0.005 inches (0.127 mm) high. The number of internal ribs 470 is preferably in the range from about 35 to about 45 equally spaced around the diameter of balloon 125.

Balloon 125 may be attached to the cannula body using any suitable bonding, heat welding or adhesive technique. A preferred construction for attaching highly compliant balloon 125 is illustrated in FIG. 4. Balloon 125, which is preferably an extruded silicone tube, is placed over the exterior of the distal body section 185 and attached proximally and distally using silicone adhesive to create interior bonds 422. One or more turns of suture material 425 may be tied or otherwise wrapped in tension around the exterior of balloon 125 in the general area of interior bonds 422. Support member 405 provides a sufficiently rigid substrate to support a tightly wound or tied suture material 425. In a preferred embodiment, suture material 425 is a polyester suture. The number of turns of suture material 425 is preferably ranges from 1 turn to about 4 turns. A final exterior layer of silicone adhesive 420 is applied over suture material 425 and the proximal and distal ends of balloon 125.

Because the multi-lumen cannula body may have a somewhat irregular or non-circular profile, it may be desirable for optimum bonding to have a separate, substantially round distal section starting from a point just distal of inflation port 430. Thus, cannula 100 may have a unitary multi-lumen construction from the proximal end to inflation port 430, and then a separate single lumen round section 455 may be placed coaxially around support member 405 having a proximal end 450 inside the area of the balloon. This allows tip section 115 to have a round profile and provides a regular, smooth surface for attaching balloon 125. Secure attachment of section 455 to support member 405 ensures a leak free assembly.

Referring to FIGS. 5 and 6, a highly compliant balloon material is more effective at achieving complete and reliable occlusion, especially when the interior of the vessel has stenotic lesions, plaque, calcifications, or other localized anomalies. FIG. 5 shows a balloon 575 that is not highly compliant expanded within an aorta 500 having a raised material 502 on the interior thereof. At a certain pressure supplied to interior space 576 within balloon 575, the balloon material is unable to satisfactorily conform and seal around raised material 502, instead leaving fluid leak paths 585. These leak paths can be closed off only by way of increased pressure to further expand the balloon and/or alter raised material 502. In contrast, FIG. 6 shows a highly compliant balloon 580 expanded within aorta 500. In this case the balloon material is able to substantially conform to and seal around the raised material 502 without increasing the pressure of the fluid within interior space 581.

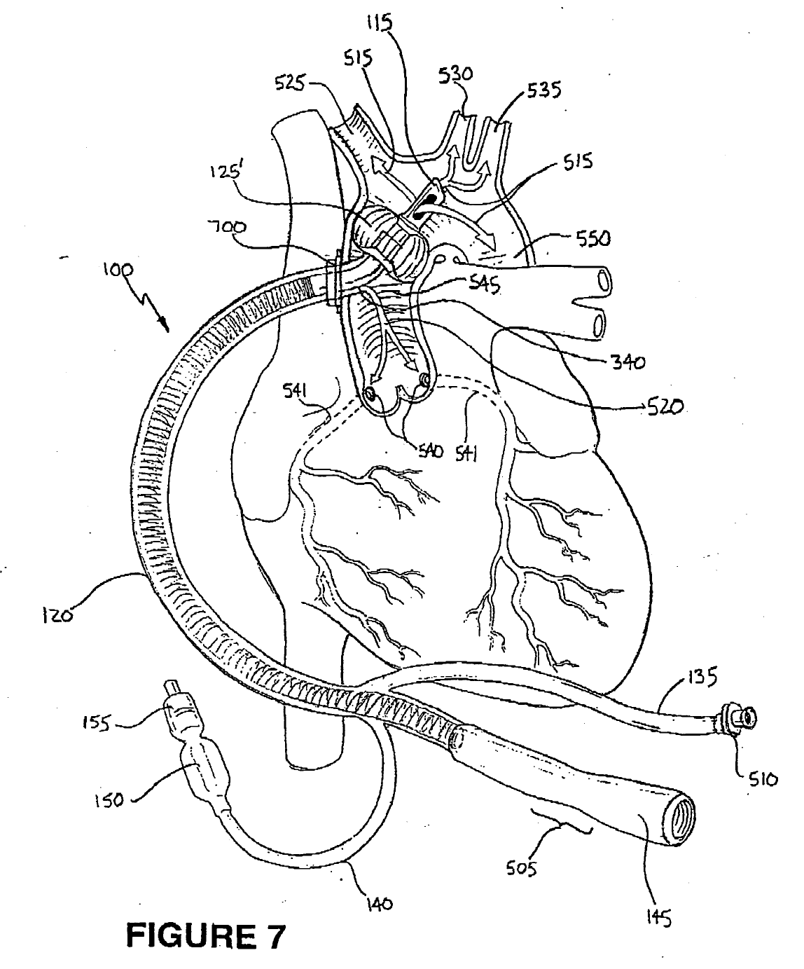

Referring to FIG. 7, cannula 100 and balloon 125 may be configured to partition the ascending aorta 545 between brachiocephalic artery 525 and coronary ostia 540. First lumen 205 is sized and configured to deliver oxygenated blood through the distal perfusion ports in the direction indicated by arrows 515, thereby supplying oxygenated blood to brachiocephalic artery 525, left carotid artery 530, left subclavian artery 535 and descending aorta 550. Second lumen 215 is sized and configured to deliver cardioplegic fluid to coronary ostia 540 which enters the myocardium antegrade through coronary arteries 541.

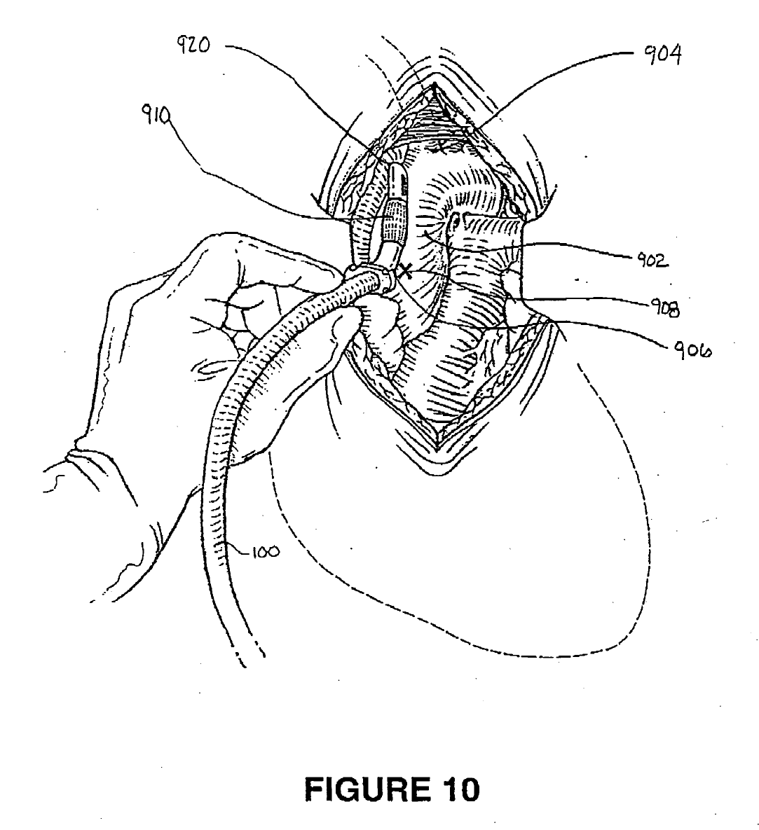

In use, the distal tip and balloon of cannula 100 are inserted through an incision in the ascending aorta as shown. Prior to making the appropriate incision in the aorta, it may be desired to place one or more purse-string sutures in the aorta to seal the incision closed against the exterior of the cannula as is generally known in the art (purse string suture 630 is illustrated, for example, in FIG. 10). To keep the operating site as uncluttered and unobstructed as possible, the ends of the purse-string sutures may be placed within a tourniquet tube and secured to slidable collar 160.

In a preferred embodiment, balloon 125 is positioned within the ascending aorta downstream of the incision. Balloon 125 may then be inflated, preferably by injecting a saline solution through connector 155 and pilot balloon 150, through inflation lumen 210, and into the interior space of balloon 125. Balloon 125 is inflated until the expanded condition of the balloon causes complete occlusion of the ascending aorta.

Inlet tube 145 is preferably clamped at a flexible section 505. Prior to connecting the oxygenated blood supply to connector 145, the clamp (not shown) is released to allow blood from ascending aorta 545 to fill first lumen 205 and thus remove all air which may have been trapped therein. Oxygenated blood may then be connected and delivered to the body through first lumen 205.

Once balloon 125 has inflated to occlude ascending aorta 545, cardioplegia may be delivered to luer connector 510, through second lumen 215, and out associated distal port 340 in the direction indicated by arrow 520. Cardioplegic solution proceeds antegrade into coronary arteries 541 and into the myocardium, thus inducing cardiac arrest. Full CPB is established as is known in the art, delivering oxygenated blood through first lumen 205 as described above. Second lumen 215 may be used to vent the area or the ascending aorta upstream of the balloon to remove any trapped air that could be delivered into the arteries as the balloon 125 is released to restore normal cardiopulmonary functions.

Markers on the exterior of the cannula may be provided to aid in proper positioning of the cannula. For example, to ensure the cannula has been inserted to the proper depth, a visible marker may be provided on the cannula body, such as marker 320 (FIG. 3) which indicates the minimum insertion depth. One or more additional markers may be provided to indicate the orientation of the distal tip relative to the cannula shaft. For example, longitudinal marker 315 (FIG. 3) indicates the direction of the distal cannula tip and balloon 125, the tip being otherwise obscured from view after insertion into ascending aorta 545.

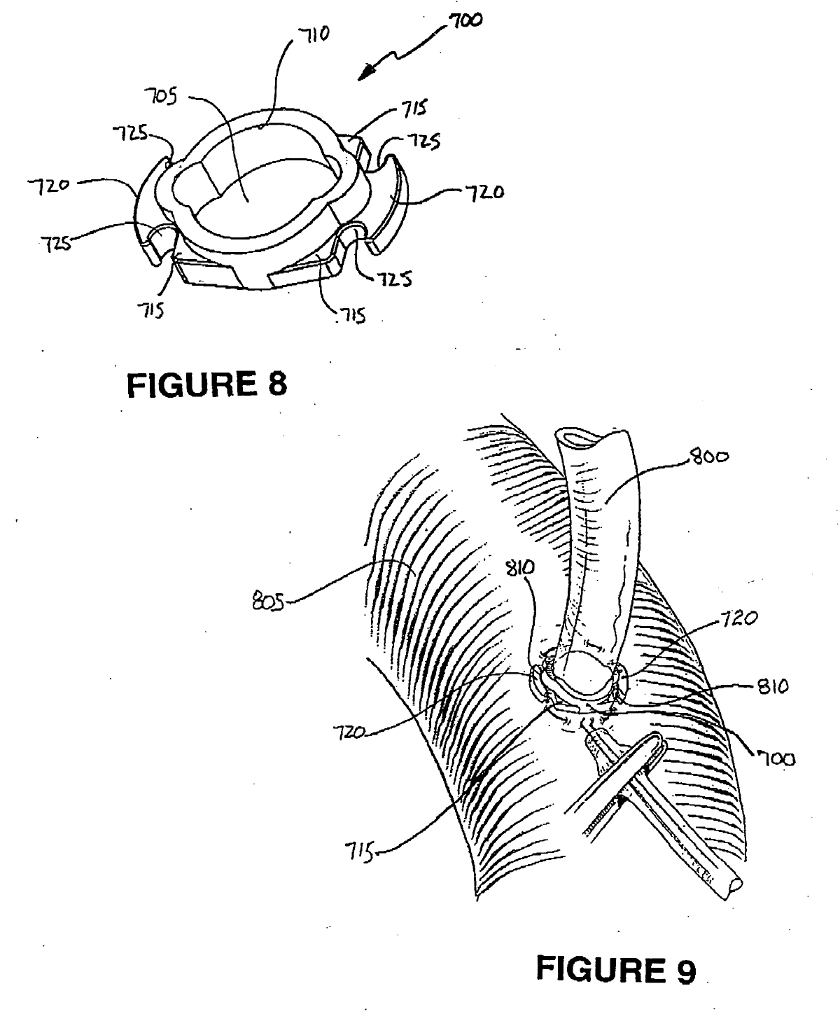

Once inserted to the proper depth within the aorta, it may be desirable to hold the cannula in place by providing features on the cannula to which the aorta can be anchored by way of sutures or the like. In one embodiment, cannula 100 may be provided with suture collar 700 which may be fastened or anchored to the aorta using sutures or other suitable fastening device. FIG. 8 further illustrates a preferred embodiment of suture collar 700 which may be attached to the main body of cannula 100.

Suture collar 700 has central bore 705 having a profile 710 shaped to correspond to the external profile of the cannula. Suture collar 700 has a stop flange 715 which may butt against the exterior of the aorta and one or more suture flanges 720 having recesses 715 for positively holding sutures from the aorta. Thus, cannula 800 having suture collar 700 can be held in a desired position relative to vessel 805 by way of sutures 810 placed through the wall of the aorta and over suture flanges 720 as shown in FIG. 9. Sutures 810 may be single, separate suture loops over each suture flange 720, or may be part of a purse string suture.

FIG. 10 illustrates a preferred method for determining the position of the incision in the aorta through which cannula 100 will be inserted. With the aorta 902 generally exposed as shown, cannula 100 is placed adjacent aorta 902 with tip 920 of cannula 100 placed at the base of the brachiocephalic or inominate artery 904. The cardioplegic port 906 on cannula 100 is visually identified and a coincident point on the aorta is marked or otherwise identified as the insertion point 908 of cannula 100. By this method, it is ensured that balloon 910 will not in any way occlude inominate artery 904 when balloon 910 is inflated to occlude aorta 902. Before making the desired incision at insertion point 908, a visual inspection should be made to ensure that there is adequate room between the insertion point 908 and the aortic root for completion of an emergency cross-clamp should one be required during the procedure and a proximal anastomosis, if required.

The various features of cannula 100, as described above, provide for reliable balloon occlusion of the ascending aorta allowing cardioplegia to be administered at a location upstream of the balloon and allowing oxygenated blood to be perfused at a location downstream of the balloon. Although cannula 100 has been illustrated to perfuse oxygenated blood generally in the area of the brachiocephalic, left carotid and left subclavian arteries (or upstream thereof), it may be desirable to perfuse the oxygenated blood at a location downstream of the aortic arch and the left subclavian artery. Such an arrangement tends to reduce the likelihood of any plaque or other particulate that may become dislodged by action of the directed flow of oxygenated blood from reaching the arteries which supply blood to the brain.

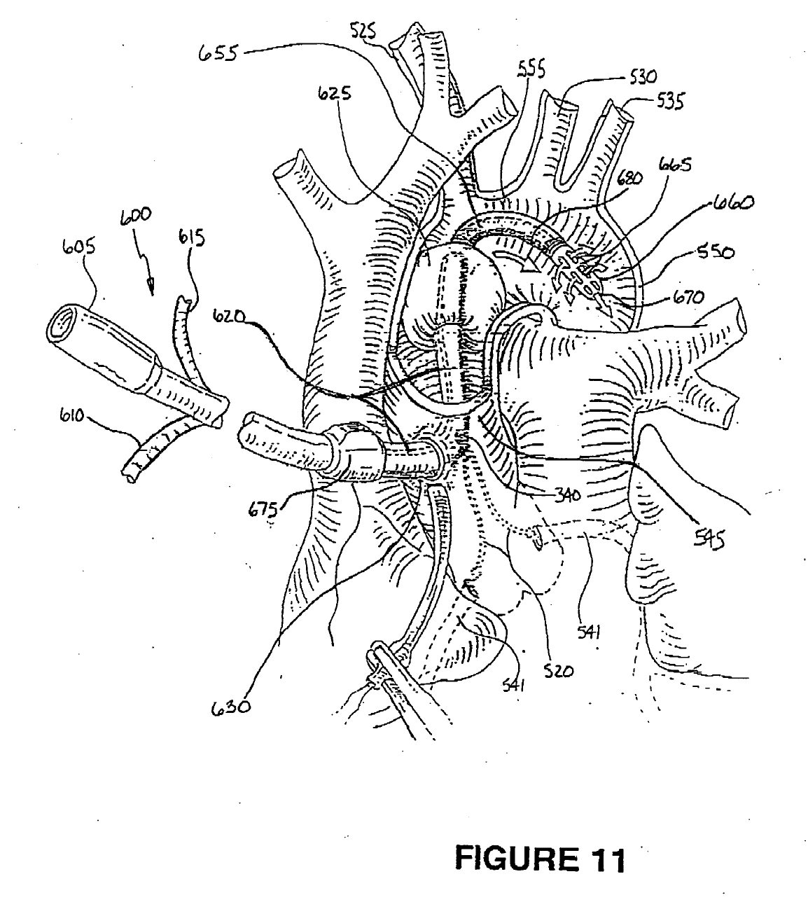

FIG. 11 illustrates a cannula configured to place the perfusion ports at a location downstream of the aortic arch. Cannula 600 has a construction similar to that of cannula 100 except that it further includes steerable tip section 655 extending distally from rigid distal section 620 which supports the balloon. Steerable tip section 655 allows the distal perfusion ports delivering oxygenated blood to atraumatically traverse past the branching arteries in aortic arch 555 and allows precise positioning of the distal perfusion ports relative to the interior wall of the aorta during pressurized perfusion of oxygenated blood.

The arrangement of the various lumen with cannula 600 are generally the same as those described above with regard to cannula 100. First lumen 205 extends through steerable tip section 655 to deliver oxygenated blood from connector 605 to the perfusion tip 660. Perfusion tip 660 has an end port 670 and one or more side ports 665. Second lumen 215 is configured to deliver cardioplegic fluid from inlet tube 610 to the coronary arteries. Balloon inflation lumen 210 is configured to deliver an inflation medium from inlet tube 615 to the interior space within the balloon. Stiff distal section 620 and steerable tip section 665 further includes a lumen for slidably receiving tension member 680, which is typically a wire or ribbon.

At or near the proximal end of rigid distal section 620 is an actuator 675 to which tension member 680 is connected. Actuator 675 may be any suitable mechanism designed to impart a sufficient force to tension member 680 to cause steerable tip section 665 to bend as desired. Suitable constructions for steerable tip section 665, tension member 680, and actuator 675 can be found, for example, in co-pending U.S. patent application Ser. No. 09/174,361, titled “CARDIOVASCULAR CANNULA WITH STEERABLE TIP”, filed on Oct. 14, 1998, the entirety of which is herein incorporated by reference.

While certain embodiments are illustrated in the drawings and have just been described herein, it will be apparent to those skilled in the art that many modifications can be made to the embodiments without departing from the inventive concepts described. It may be desirable, for example, to include one or more additional lumen to the cannulae described above for any number of purposes known in the art. It may be desirable, for instance, to include one or more pressure monitoring lumen for monitoring aortic pressures upstream or downstream of occlusion balloon 125.

Further, for purposes of illustration only, the principles of the present invention has been described primarily with reference to CPB procedures but may readily be applied to other types procedures not specifically described. Many other uses may be known in the art, and the concepts described herein are equally applicable to those other uses. Further, the different components of the various exemplar embodiments described above can be combined in any desirable construction. Accordingly, the invention is not to be restricted except by the claims which follow.

What is claimed is:

1. A cannula device for delivering oxygenated blood and cardioplegia to the ascending aorta, said cannula device comprising:
an elongated tubular body having a proximal portion and a substantially rigid distal portion, said proximal portion having a longitudinal axis, and said distal portion having a section thereof disposed at an angle relative to said longitudinal axis;
an inflatable balloon attached to said section; and
a first lumen extending from said proximal portion to an open port distal to said balloon, a second lumen extending from said proximal portion to an open port proximal to said balloon, and an inflation lumen in fluid communication with said inflatable balloon.

2. The cannula device of claim 1 wherein said substantially rigid distal portion includes a support tube.

3. The cannula device of claim 2 wherein said rigid support is made of metal.

4. The cannula device of claim 1 wherein said angle is between about 100 degrees to about 120 degrees.

5. The cannula device of claim 1, wherein said proximal portion further includes a coiled wire support member surrounding at least a portion of said first lumen.

6. The cannula device of claim 1, wherein said balloon is made from a material which is capable of an elastic elongation of at least 500%

7. The cannula device of claim 6, wherein said balloon is silicone.

8. The cannula device of claim 1, wherein said balloon is a tubular member attached to said section at a first area and a second area proximal to said first area, said cannula device further comprising a suture material wrapped at least once around said tubular member at said first area and said second area.

9. The cannula device of claim 1 wherein said balloon is a tubular member having an interior surface, said interior surface having a plurality of raised ribs.

10. The cannula device of claim 1 wherein said elongated tubular body further comprises at least one flange for securing a suture attached to the aorta.

11. A cannula device for partitioning a vessel within a body into an upstream portion and a downstream portion, said cannula device comprising:
an elongated tubular body having a proximal portion and a distal portion, said proximal portion having a longitudinal axis, and said distal portion having a section thereof disposed at an angle relative to said longitudinal axis;
an inflatable balloon attached to said section, said balloon made from a material which is capable of an elastic elongation of at least 700%; and
a first lumen having an open port distal to said inflatable balloon, a second lumen having an open port proximal to said inflatable balloon, and an inflation lumen in fluid communication with said inflatable balloon.

12. The cannula device of claim 11, wherein said distal portion includes a rigid support tube.

13. The cannula device of claim 11 wherein said inflatable balloon material is silicone.

14. The cannula device of claim 11, wherein said angle is between about 100 degrees and about 120 degrees.

15. A cannula device for delivering oxygenated blood and cardioplegia to the aorta, said cannula device comprising:
an elongated tubular body having a proximal portion, a distal portion, a first lumen for delivering oxygenated blood to the aorta and a second lumen for delivering cardioplegia to the aorta;
said distal portion having a substantially rigid support member having an inner surface and an outer surface, said distal portion having a layer of polymeric material disposed over said outer surface of said support member, said polymeric material having an exterior surface; and
and an inflatable balloon attached to said exterior surface of said polymeric layer.

16. The cannula device of claim 15 wherein said proximal portion is flexible.

17. The cannula device of claim 15 wherein at least a portion of said proximal portion further comprises a coil member disposed within said first lumen.

18. The cannula device of claim 17 further comprising a polymeric coating substantially covering said inner surface and said coil member.

19. The cannula device of claim 15 wherein said inflatable balloon and said polymeric layer are made from silicone.

20. The cannula device of claim 15 wherein said inflatable balloon is tube shaped having a proximal end and a distal end, and wherein said inflatable balloon is attached to said exterior surface using one or more sutures fixed around one or both of said proximal and said distal ends of said balloon.
